(19) United States
(12) Patent Application Publication
(10) Pub. No.: US 2010/0280338 A1
(43) Pub. Date: Nov. 4, 2010

(54) EAR-WORN BIOFEEDBACK DEVICE

(76) Inventor: Chang-An Chou, Taipei (TW)

Correspondence Address: Chang-An Chou, 3F, No. 100, Sec. 3. Mingsheng E. Rd., Taipei, 105, TW

(21) Appl. No.: 12/810,514

(22) PCT Filed: Dec. 29, 2008
(86) PCT No.: PCT/CN08/02102

§ 371 Date: Jun. 25, 2010

(30) Foreign Application Priority Data
Dec. 28, 2007 (CN) 200710306614.2

(51) Int. Cl. A61B 5/00

(52) U.S. Cl. 600/301

(57) ABSTRACT

An ear-worn biofeedback device is provided including an ear-worn structure for mounting on at least an ear of the user, at least a housing attached to the ear-worn structure, at least a sensor/electrode, for acquiring biosignals, and a circuit system accommodated in the housing. The circuit system includes an analog/digital converter for receiving and digitizing biosignals acquired by the sensor/electrode, a processor, a battery, for providing power, and a sound module, disposed around the ear, wherein during the biofeedback process, the digitized biosignals are analyzed, and when a preset condition is matched, the processor notifies the user by driving the sound module to generate audio signals for being received by the ear.

### FIELD OF THE INVENTION

The present invention is related to an ear-worn biofeedback device, and more particularly to an ear-worn biofeedback device which provides user-friendly installation and intuitive operation procedure.

### BACKGROUND OF THE INVENTION

Biofeedback is a form of alternative medicine that involves in measuring a subject's quantifiable bodily functions such as blood pressure, heart rate, skin temperature, sweat gland activity, and muscle tension, and conveying the information to the patient in real-time in visual (such as, figures, light) or audio fashion, so as to raise the user's awareness and conscious control of their unconscious physiological activities.

One kind of biofeedback is neurofeedback/EEG biofeedback, which utilizes measured brain wave information to train the user's own central nervous system, namely, to change his/her own brain waves. The neurofeedback is based on the scientifically proven theory that under different predominant brain waves, people present different behaviors, motions, and physiological conditions.

Nowadays, brain waves are mainly divided into δ wave (0˜4 Hz), θ wave (4˜8 Hz), α wave (8˜12 Hz), SMR (sensory-motor rhythm) (12˜15 Hz), β wave (15˜28 Hz) and γ wave (28˜70 Hz and up).

δ wave, also called slow wave, is the predominant brain wave during deep sleep or unconscious, usually stages three and four of NREM (non-rapid eye movement) sleep, so that δ wave is relative to sleep quality. θ wave can be seen in drowsiness or arousal and also in meditation, and at this stage, memory storage can be enhanced. α wave is brought out by closing the eyes and by relaxation. SMR (sensory-motor rhythm) is produced when the corresponding sensory-motor areas are idle, e.g. during states of immobility. SMR typically decreases in amplitude when the corresponding sensory or motor areas are activated, e.g. during motor tasks and even during motor imagery. β wave is associated with normal waking consciousness and can be further divided into mid-beta wave, 15˜22 Hz, and hi-beta wave, 22˜28 Hz. γ wave is produced when awakening and REM stage, and is involved in higher mental activity. Transient periods of synchronized firing over the γ waveband, of entire banks of neurons from different parts of the brain, have been proposed as a mechanism for bringing a distributed matrix of cognitive processes together to generate a coherent, concerted cognitive act, such as perception.

Modern medical science has proven that people can change their own brain waves via neurofeedback so as to improve or treat many mental disorders, such as, epilepsy, melancholia, manic-depression, ADD (Attention Deficit Disorder), and ADHD (Attention Deficit with Hyperactivity Disorder). For example, melancholia is associated with α wave distribution in right brain and left brain, and people who feel depression will have more α wave distribution in right brain. Therefore, through brain wave detection and biofeedback process, the symptom can be eased significantly. On the other hand, it also discovered that ADHD patient's brain produces more θ waves and less SMR waves, so that through neurofeedback, it can increase the production of θ waves, and inhibit the production of SMR wares.

Hence, more and more devices, systems involving in biofeedback are recently developed.

The configuration of common biofeedback device is: the user sits in front of a computer and connects with various biosensing elements from the computer for examining, for example, oxygen saturation and/or GSR (galvanic skin response); then, program preloaded in the computer is executed to calculate physiological parameters related to biofeedback; and according to the calculated parameters, an audio and/or visual response is fed back to the user via the computer, so as to influence the physiological conditions. For example, U.S. Pat. No. 6,652,470 is related to diagnose and treat ADHD by biofeedback, in which the computer is used as the feedback interface for achieving the biofeedback effect on the user sitting in front of the computer.

Moreover, in considering the using convenience, the biofeedback device is developed to be portable. For example, WO 2006/113900 discloses a handheld stress relieving device. The relieving mechanism thereof depends on controlled respiration can shift the balance between the sympathetic and parasympathetic branches. In general, reducing breath frequency, increasing tidal volume, and/or increasing the expiration/inspiration ratio can increase parasympathetic activity so as to elicit the relaxation response, and further, respiratory sinus arrhythmia (RSA) waves, resting HRV (Heart rate variability) waves, which are related to the rate, rhythm and depth of a person's breathing, can be calculated from pulse rates. Therefore, this stress relieving device detects user's pulse rates from the inserted finger, provides user with information on his or her RSA waves, and utilizes breathing guide on a display (which alters according to the variation of the detected pulse rates) to instruct the users to control their respiration frequency, so as to achieve the effect of stress relieving.

The above describes the current biofeedback devices. However, these conventional biofeedback devices are still incapable of satisfying the following requirements:

1. Requirement for reducing external interference: As executing biofeedback, if the external noise can not be lowered down, especially in a noisy environment, the expected effect, such as, stress relieving, might not be achieved.

2. Requirement for manners other than visual feedback: When the biofeedback purpose is to relax, visual feedback might hinder the usual relaxing behaviors, such as, closing eyes.

3. Requirement for privacy: When in a public environment, such as, public transportation system, the feedback information shown on the handheld screen might embarrass the user since any one nearby can see the contents on the screen.

4. Requirement for convenience: If the biofeedback device has to be held by hand, the user is therefore restricted by occupied hand.

Therefore, there is the need to provide user a novel biofeedback device which can fulfill the above-described requirements.

The object of the present invention is to provide an ear-worn biofeedback device which utilizes an audio feedback manner to avoid manual biofeedback process, so as to enhance feasibility.

Another object of the present invention is to provide an ear-worn biofeedback device which can effectively reduce the external interferences through the fashion of ear-worn, thereby providing user a more concentrated operating condition.

### SUMMARY OF THE INVENTION

In one aspect of the present invention, the present invention provides an ear-worn biofeedback device including an ear-worn structure for mounting on at least an ear of the user, at least a housing attached to the ear-worn structure, at least a sensor/electrode for acquiring biosignals, and a circuit system accommodated in the housing. The circuit system further includes an analog/digital converter for receiving and digitizing biosignals acquired by the sensor/electrode, a processor, a battery, for providing power, and a sound module disposed around the ear. Furthermore, during the biofeedback process, the digitized biosignals are analyzed, and when the preset condition is matched, the processor notifies the user by driving the sound module to generate audio signals for being received by the ear.

According to the description above, the audio signals can be generated continuously or intermittently, and can be in various forms, e.g., soft, rushed, so as to provide different feedback effects, for example, for guiding the user to change from a current physiological condition to another physiological condition, for driving the user to enter a physiological condition, or for notifying the user a physiological condition is achieved.

In a preferred embodiment, the ear-worn structure can also be implemented to accommodate part of the circuit system, or the electric connection for the circuit system especially when the circuit system is separated to locate in two housings.

Moreover, the ear-worn structure can be implemented as a single or dual ear-worn structure for mounting on one or two ear(s) of the user. And, if the ear-worn structure is insufficient for fixing or accommodation, a connecting element can be further included no matter for placing the circuit system/electric connection and/or for assisting in fixing the whole device.

Preferably, the ear-worn biofeedback device of the present invention is implemented to have a wireless module, so that it can wirelessly communicate with external device, for example, computer device, mobile phone, PDA, display device, another biosignal acquisition device, or another biofeedback device. Therefore, in addition to the processor executes the analysis of the biosignals acquired by the sensor/electrode, the biosignals also can be analyzed by the external device through the wireless output, and then transmitted back to the processor for the following matching process, so that even a more advanced calculation or analysis can be performed without sacrificing the simplicity and compactness of the device. And, if the external device has the function of display, then the visual feedback can be performed, and thus, the present invention can be expanded as a biofeedback device with both visual and audio feedbacks.

Besides, through wireless communication, the ear-worn biofeedback device can also receive information from the external device, especially the biosignals acquired by another biosignal acquisition device, so that a more accurate analysis can be achieved by comparing more kinds of biosignals. In a preferred embodiment, the external device can be implemented as another biofeedback device with compatible wireless module, so that via the wireless communication therebetween, two users, nearby or faraway, can have a real-time interaction, with or without mediated computer and/or network, by respective biofeedback mechanisms, for example, for playing game, for competition.

In another preferred embodiment, when the external device is a remote server operated by medical personnel which are communicated through the network, a remote real-time response from the medical personnel can be transmitted back to the user, and particularly, through the sound module provided by the present invention, an audio indication also can be transmitted to the user without limitation.

In another preferred embodiment, the ear-worn biofeedback device can have two sound modules for respectively disposing around both ears of the user, so that a biofeedback process relating both sides of brain can be performed.

### BRIEF DESCRIPTION OF THE DRAWINGS

FIGS. 1A˜1B are schematic views showing the structure of the ear-worn biofeedback device in a preferred embodiment of the present invention and the combination with the ear;

FIG. 2 is a circuit block showing the circuit system of the ear-worn biofeedback device according to the present invention;

FIG. 3A is a schematic view showing the ear-worn biofeedback device implemented into a headphone structure according to a preferred embodiment of the present invention;

FIG. 3B is a schematic view showing the ear-worn biofeedback device implemented into a neckband headphone structure according to a preferred embodiment of the present invention;

FIG. 4 is a schematic view showing the ear-worn biofeedback device wirelessly communicated with an external biosignal acquisition device in a preferred embodiment according to the present invention;

FIG. 5 is a schematic view showing the ear-worn biofeedback device implemented to function as the BCI (Brain Computer Interface) in a preferred embodiment according to the present invention; and

FIG. 6 is a schematic view showing the ear-worn biofeedback device wirelessly communicated with an external handheld device in a preferred embodiment according to the present invention.

### DETAILED DESCRIPTION OF THE PREFERRED EMBODIMENTS

First, the reasons why the ear-worn form is selected are:

1. Although sounds and images are both workable and great biofeedback choices, the visual biofeedback typically requires the user to keep eyes open, which might increase the difficulty in relaxing, and oppositely, the audio biofeedback provides a more natural way with less restriction.

2. As compared with the biofeedback device acquiring biosignals from hand (such as, WO 2006/113900), the ear-worn biofeedback device can directly acquire biosignals from ear(s) without hand occupation.

3. When the biofeedback device is held by hand, sound is still the main feedback medium, but without earphone, people nearby might be disturbed. Therefore, if the device can be directly worn by ear(s), the sound can be limited around the ear(s) without influencing others, and the inconvenience of connecting wire as using earphone can also be eliminated.

4. The ear-worn device also provides the function of shading and/or isolation, which lowers down the influence of external interference, such as, noises, talks.

5. The ear-worn device provides a using pattern similar to the general purpose earphone/headphone, so that, even in the public environment, the user will still feel graceful and comfortable.

Therefore, through the ear-worn device, in addition to carrying convenience, the user also can have a comfortable using experience in the public environment, especially MP3 player has become so popular that a lot of people wear earphone as in the public transportation system or walking, so that the ear-worn form will not embarrass the user. Even, the ear-worn biofeedback device can be combined with the MP3 Player for increasing functionality.

Besides, the ear-worn type is also advantageous that many biosignals can be acquired through the ears, for example, hear rate, blood oxygen saturation, blood pressure, and EEG, and some other biofeedback-related biosignals can be acquired in the vicinity of ear(s), for example, respiration, EOG and facial EMG, so that through placing the device around the ear, the wiring complexity can have a significant reduction, thereby improving using convenience.

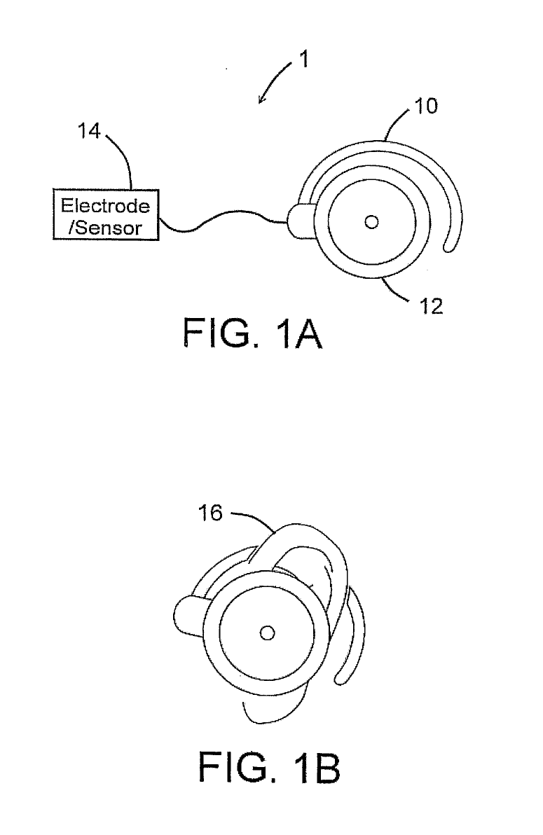

Please refer to FIG. 1A˜1B, which are schematic views showing the structure of the present invention and the combination with the ear. As shown, the ear-worn biofeedback device 1 according to the present invention includes an ear-worn structure 10, a housing 12, and at least a sensor/electrode 14, wherein the housing 12 has, accommodated therein, a circuit system connected with the sensor/electrode 14. The ear-worn structure 10 is used for supporting the whole device by bearing the housing 12 and the circuit system therein and is used to mount on at least an ear 16 of the user. Here, there is no limitation to the implementation of the ear-worn structure 10, and the only requirements thereof are to bear the housing 12 and the circuit system and mount on the ear(s) 16, for example, it can be a single ear-worn or dual ear-worn structure, and it can be a headphone-like structure, an in-ear structure, a neckband headphone-like structure, a clip-on earphone-like structure or even a cap or headdress. Therefore, the implementation of the ear-worn structure can be varied as demand changes.

As to the sensor/electrode 14, it is also not restricted, that is, those can acquire biosignals that are related to biofeedback all can be used in the present invention, for example, physiological electrodes, e.g., EEG electrode, EOG electrode, EKG electrode, and EMG electrode, and physiological sensors, e.g., temperature sensor, and optical sensing elements which might use to detect cardiovascular biosignals (such as, blood oxygen saturation and blood pressure). Hence, the type and the quantity of sensor/electrode both are not limited.

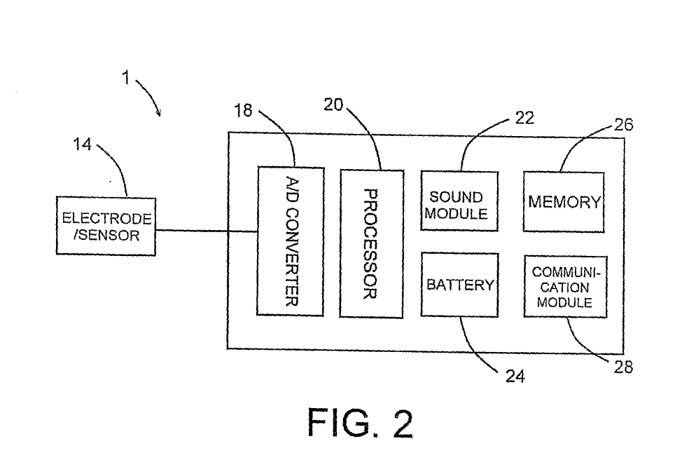

Now, please refer to FIG. 2, which is a circuit block showing the circuit system of the present invention. As shown, the circuit system includes an A/D (analog-to-digital) converter 18, a processor 20, a sound module 22 and a battery 24, wherein the A/D converter 18 is used to receive and digitize biosignals acquired by the sensor/electrode 14, then the digitized biosignals are transmitted to the processor 20 and analyzed by a program preloaded in the processor 20 to know if the biosignals match to a preset condition, and when the preset condition is matched, the processor 20 drives the sound module 22 to generate audio signals, so as to notify the user. Here, because the sound module 22 is disposed around the ear(s), the audio signals can be directly received by the ear(s), and after receiving the audio signals, the user can have a response according thereto. Then, the above-described procedures are continuously repeated until an expect biofeedback effect is achieved, such as, the stress is relieved or the brain waves have changed.

According to the present invention, the audio signals are used to guide the user to perform a proper action, for example, change breathing frequency or be concentrated, for gradually completing the biofeedback process. Therefore, the audio signals can have diverse implementations for indicating different situations. For example, when the analysis result and the preset condition are not matched, the audio signals can be presented as short and rushed, and when the analysis result is getting close to the preset condition, the audio signals can gradually become softer and slower. Or, the audio signals can be used to inform the user that what kind of physiological condition he/she has achieved. Or, the audio signals can be generated to drive the user to enter a physiological condition, or to drive the user to change from a current physiological condition to another physiological condition. Moreover, the type of audio signal also can be different. For example, if the audio signals are used in a stress-relieving biofeedback, soft and gentle sounds can be selected. Further, the audio signals also can be generated continuously or intermittently depending on situation variances. Therefore, in accordance with different operation purposes and different physiological conditions, the audio signal types can be varied without limitation.

Furthermore, the circuit system also includes a memory 26 for storing physiological information and related data, so that in addition to the real-time biofeedback, the biosignals and physiological conditions also can be recorded for facilitating a further analysis. For example, the user can download the recorded data to a computer and view the detailed records, or the doctor can utilize the recorded data to get more understanding of patient's physiological variations. Here, the memory can be implemented as a built-in memory or a removable memory, such as, SD card, there is no limitation.

Besides, the circuit system further includes a communication module 28 for achieving a communication with an external device. Here, the communication module can be used to perform wired or wireless transmission. For example, USB connection is the well-known wired communication for data output and input, so that when there is a need to change the biofeedback process for adapting different symptoms, the program can be uploaded through USB connection or the external device can change the settings of the biofeedback device through USB connection.

Then, if the communication module 28 is implemented to perform wireless transmission, not only the functions described above can be achieved, more benefits also can be obtained. For example, during the biofeedback process, the ear-worn biofeedback device can wirelessly receive biosignals from another biosignal acquisition device, so that the biosignals acquired by the sensor/electrode 14 and the wirelessly received biosignals can be analyzed together, thereby achieving a more accurate biofeedback effect since more kinds of biosignals are involved in calculation and analysis. Or, the ear-worn device also can wirelessly transmit the acquired biosignals and/or the analysis result to an external device, such as, a computer, a handheld device, another biosignal acquisition device, or another biofeedback device, for displaying, processing and/or storage, and if the external device is capable of connecting to a network, such as, Internet, then the biofeedback device even can be communicated with a remote device, e.g., a computer of a doctor, or a server, so that the user can have an instant communication with a remote doctor, and further, via the sound module, the doctor can instruct the user directly.

Moreover, via the wireless module 28, the biosignals acquired by the sensor/electrode 14 also can be wirelessly transmitted to the external device for calculation and analysis, that is, the acquired biosignals are processed in the external device (such as, computer, PDA, handheld device, or another biosignal acquisition device), and then the processed result is transmitted back to the biofeedback device, so that the processor 20 can decide if the result matches to the preset condition, and drive the sound module to generate audio signals. Therefore, through the external device, a more advanced calculation and analysis can be performed without sacrificing the compactness and the simplicity of the device. Here, the external device also can be used to display related physiological information, so as to additional complete a visual feedback process.

Furthermore, through wireless communication, a more interesting operation also can be achieved. For example, multiple biofeedback devices can have a real-time interaction thereamong, so that the users of respective biofeedback devices can execute, e.g., a mind-controlled game using EEG signals. And, further through the network, a biofeedback device can be communicated with at least a remote biofeedback device, for example, by commonly communicating with a computer device connecting to the network or by directly connecting to the network, and thus, an on-line game using biofeedback signals is further achieved.

That is, through the wireless communication, the biofeedback device according to the present invention can be expanded simply and easily without wiring and carrying burden.

Following are some examples illustrating the ear-worn biofeedback device according to the present invention, but it should be understood that these are only partial embodiments and the present invention is not restricted thereby.

### Example I

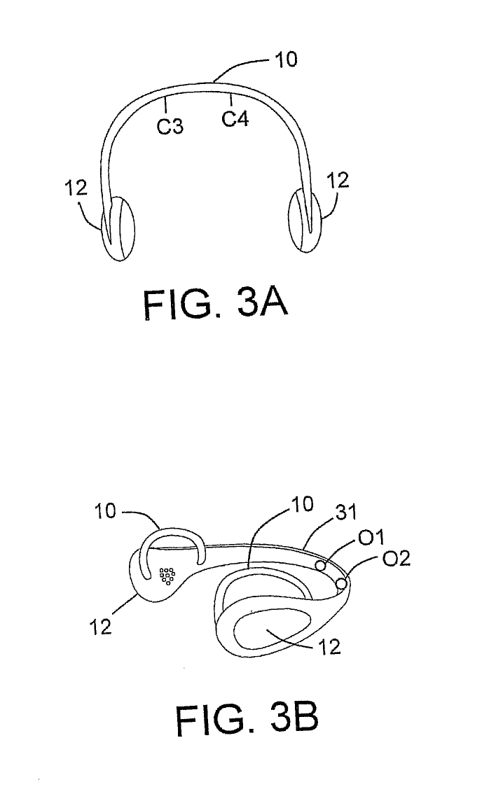

As shown in FIGS. 3A˜3B, the ear-worn biofeedback device of the present invention is implemented as a dual ear-worn form. In this embodiment, due to the dual ear-worn structure, two housings 12 are employed for respectively placing around two ears of the user. In FIG. 3A, the electric connection between two housings 12 is directly located in the ear-worn structure 10, which just looks like the general-used headphone, and in FIG. 3B, a connecting element 31 is further employed for accommodating the electric connection, which is similar to the common neckband headphone.

These two embodiments are particularly suitable for EEG detection since other than A1, A1 EEG signals on the ears or GROUND on mastoid, C3, C4 (FIG. 3A) or O1, O2 EEG signals also can be acquired by properly positioning the ear-worn structure and/or the connecting structure, so that a simple mode EEG detection can be provided.

At this time, if both housings 12 have the sound module mounted therein, then it will be a possible design for realizing the different actions between left and right brains, thereby the balance training, for example, can be performed.

As to the arrangement of circuit system, it can be mounted in one housing, or separated in two housings, and if the circuit system is separated, the electric connection therebetween can be, as described above, accommodated in the ear-worn structure and/or the further employed connecting element for reducing wiring complexity. Besides, the sound module also can be selected to mount in one or two housing(s). The whole configuration is flexible and is capable of being varied according to different demands, and/or the considering of manufacturing cost.

### Example II

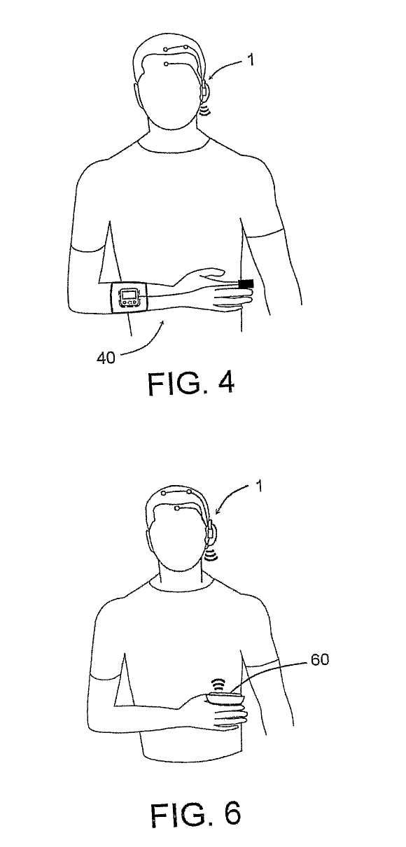

Since the ear-worn biofeedback device is located around the ear(s), it is convenient to acquire oxygen saturation and heart rate from the ear(s) at the same time. Then, as shown in FIG. 4, if the ear-worn biofeedback device 1 can be further wirelessly communicated with a GSR (Galvanic skin response) meter 40, the whole system can be used to perform the biofeedback process for ANS (Autonomic Nervous System).

The ANS biofeedback mechanism is that through controlling breathing, thought and consciousness, a feedback to the autonomic nervous system can be produced, thereby indirectly accomplishing the control to the autonomic nervous system. In this embodiment, the oxygen saturation, heart rate and GSR are used to judge the predomination of sympathetic nerve system or parasympathetic nerve system, and according to the judgment, the biofeedback device can guide the user to adjust his/her physiological conditions. For example, when the sympathetic nerve system is predominant, it means the body is under hyperfunction, such as, faster heart beat, hurried breathing, and sweat hands, and when the parasympathetic nerve system is predominant, the body will be in a relaxed status, e.g., slower heart beat, smooth breathing and dry hands. Therefore, by understanding the autonomic nervous system, it can guide the user to change breathing frequency or thought, so as to further influence the autonomic nerve system.

### Example III

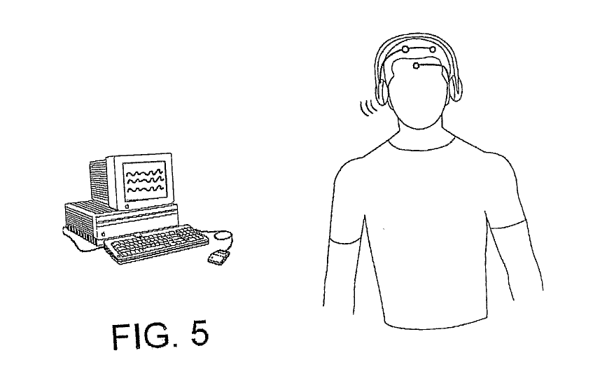

As shown in FIG. 5, the ear-worn biofeedback device according to the present invention also can be used to be the BCI (Brain Computer Interface). As shown, the ear-worn biofeedback device employs the wireless module to wirelessly transmit biosignals, especially EEG biosignals, to a computer with the corresponding (built-in or externally connected) receiver (not shown), so that the mechanism for controlling the computer by thought is completed. Through this configuration, no matter the training of concentration or game control, all can be achieved. For example, multiple biofeedback devices can be wirelessly communicated with each other or simultaneously connected with a computer for playing game, and even, the biofeedback device can communicated with another remote biofeedback device via network.

### Example IV

According to one embodiment of the present invention, in addition to employing the audio signals to guide the user, a device 60 with display function also can be provided, as shown in FIG. 6, for simultaneously utilizing visual feedback mechanism. Here, the device 60 with display function can be a handheld device, such as, a mobile phone, a MP3/MP4 player, and a PDA, or a wrist-worn device, such as, a watch, or simply a display device, or even can be another biosignal acquisition device. Besides, the device 60 also can provide other functions, such as, further analysis, storage, and/or networking, so as to expand the usable range of the ear-worn biofeedback device.

In the aforesaid, through combining with selectable sensors/electrodes and cooperating with external devices of different functions, the ear-worn biofeedback device according to the present invention can be implemented into different biofeedback devices for various purposes, for example, epilepsy alarm, drowsiness alarm, ADD/ADHD treatment, PTSD (Post-Traumatic Stress Disorder) treatment, memory enhancement, ANS (Autonomic Nervous System) adjustment, apoplexy rehabilitation, medicine/alcohol withdrawal, body enhancement (e.g., athletes), decision making ability enhancement, medical training, brain state driving, stress relieving etc. Therefore, the ear-worn form implementation of biofeedback device according to the present invention can be widely applied to numerous biofeedback mechanisms.

Moreover, the ear-worn structure not only provides an earphone-like, ergonomic design, just like the normal earphone, headphone, neckband headphone, clip-on earphone, or Bluetooth earphone for mobile, which will not make the user feel uncomfortable or embarrassed, but also approves a simple and wearable design which allows easily operating in various environments without burden. Furthermore, by the ear-worn form, the biosignals from the ear(s) can be easily acquired without complicated wiring, and simultaneously, the external interference, noise can be reduced. And, the all-in-one design provides great integrity which facilitates user's operation. Consequently, the present invention provides a convenient biofeedback device which achieves not only better portability, but also intuitive procedure.

What is claimed is:

1. An ear-worn biofeedback device, comprising:
an ear-worn structure, for mounting on at least an ear of the user;
at least a housing, attached to the ear-worn structure;
at least a sensor/electrode, for acquiring biosignals; and
a circuit system, accommodated in the housing, comprising:
an analog/digital converter, for receiving and digitizing biosignals acquired by the sensor/electrode;
a processor;
a battery, for providing power; and
a sound module, disposed around the ear,
wherein
during the biofeedback process, the digitized biosignals are analyzed, and when a preset condition is matched, the processor notifies the user by driving the sound module to generate audio signals for being received by the ear.

2. The device as claimed in claim 1, wherein the audio signals are generated continuously or intermittently.

3. The device as claimed in claim 1, wherein the audio signals are used to guide the user to change from a current physiological condition to another physiological condition, to drive the user to enter a physiological condition, or to notify the user a physiological condition is achieved.

4. The device as claimed in claim 1, wherein the analysis of the biosignals is performed by the processor.

5. The device as claimed in claim 1, further comprising a wireless module for performing wirelessly transmission.

6. The device as claimed in claim 5, wherein the wireless module wirelessly receives biosignals from a biosignal acquisition device for being analyzed by the processor.

7. The device as claimed in claim 6, wherein the analysis of the received biosignals is implemented together with the biosignals acquired by the sensor/electrode.

8. The device as claimed in claim 5, wherein the biofeedback device is wirelessly communicated with an external device through the wireless module.

9. The device as claimed in claim 8, wherein the external device wirelessly receives the biosignals for further analysis, and after analysis, the analysis result is transmitted back to the biofeedback device.

10. The device as claimed in claim 8, wherein the external device wirelessly receives the biosignals for displaying and/or storage.

11. The device as claimed in claim 8, wherein the external device is a handheld device or a wrist-worn device.

12. The device as claimed in claim 8, wherein the external device is a mobile phone, a PDA or a display device.

13. The device as claimed in claim 8, wherein the external device is a second biofeedback device used by another user.

14. The device as claimed in claim 13, wherein the biofeedback device and the second biofeedback device have a real-time interaction therebetween.

15. The device as claimed in claim 14, wherein the real-time interaction is used for achieving a game.

16. The device as claimed in claim 13, wherein the biofeedback device and the second biofeedback device achieve a wireless communication therebetween by having a compatible pair of wireless modules, by commonly connecting to a computer device, or by further connecting to a network.

17. The device as claimed in claim 8, wherein the external device is capable of connecting the biofeedback device to a network.

18. The device as claimed in claim 17, wherein the network is Internet.

19. The device as claimed in claim 17, wherein the biofeedback device is connected to a remote device through the network.

20. The device as claimed in claim 19, wherein the remote device is a server.

21. The device as claimed in claim 19, wherein the remote device is operated by medical personnel.

22. The device as claimed in claim 5, wherein based on the wireless module, the biofeedback device is configured and/or the biofeedback device executes the data upload/download.

23. The device as claimed in claim 1, wherein the sensor/electrode is at least one selected from a group consisting of: physiological electrode, temperature sensing element, and optical sensing element.

24. The device as claimed in claim 1, wherein the ear-worn structure is a single ear-worn structure, or a dual ear-worn structure.

25. The device as claimed in claim 1, wherein the circuit system further includes a memory for storing the biosignals and/or the analysis result.

26. The device as claimed in claim 25, wherein the memory is removable memory.
